(19) United States
(12) Patent Application Publication
(10) Pub. No.: US 2017/0221262 A1
(43) Pub. Date: Aug. 3, 2017

(54) MEDICAL DEVICES FOR MAPPING CARDIAC TISSUE AND METHODS FOR DISPLAYING MAPPING DATA

(71) Applicant: Boston Scientific Scimed Inc., Maple Grove, MN (US)

(72) Inventors: Jacob I. Laughner, St. Paul, MN (US); Shibaji Shome, Arden Hills, MN (US); Kevin J. Stalsberg, White Bear Lake, MN (US); Scott A. Meyer, Lakeville, MN (US)

(21) Appl. No.: 15/490,847

(22) Filed: Apr. 18, 2017

Related U.S. Application Data
(63) Continuation of application No. 14/631,055, filed on Feb. 25, 2015.
(60) Provisional application No. 61/949,081, filed on Mar. 6, 2014.

(51) Int. Cl. G06T 17/20; A61B 5/042; A61B 5/0452; A61B 5/04; A61B 90/92; A61B 5/044; A61B 5/00

(52) U.S. Cl. CPC G06T 17/20; A61B 5/044; A61B 5/0422; A61B 5/6858; A61B 5/04012; A61B 90/92; A61B 5/04011; A61B 5/0452; G06T 2210/41; A61B 2562/0209

(57) ABSTRACT

Methods for displaying physiological mapping data are disclosed. An example method may include storing a set of three-dimensional positional data on a memory, storing a set of metric data on the memory, and storing a set of electrogram data on the memory. The method may also include outputting the set of three-dimensional positional data, the set of two-dimensional metric data, and the set of electrogram data from the memory to a display unit and displaying the set of three-dimensional positional data, the set of two-dimensional metric data, and the set of electrogram data on the display unit as a dynamic display.

### CROSS-REFERENCE TO RELATED APPLICATIONS

This application is a continuation of U.S. application Ser. No. 14/631,055, filed Feb. 25, 2015, which claims priority under 35 U.S.C. §119 to U.S. Provisional Application Ser. No. 61/949,081, filed Mar. 6, 2014, all of which are incorporated herein by reference.

### TECHNICAL FIELD

The present disclosure pertains to medical devices, and methods for using medical devices. More particularly, the present disclosure pertains to medical devices for mapping cardiac tissue and methods for displaying mapping data.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices. An example method for displaying physiological mapping data is disclosed. The method includes storing a set of three-dimensional positional data on a memory. The set of three-dimensional positional data corresponds to a location of one or more electrodes within a body chamber. The method also includes storing a set of metric data on the memory. The set of metric data corresponds to a pre-determined metric collected by the one or more electrodes. The method also includes storing a set of electrogram data on the memory. The set of electrogram data corresponds to electrical activity sensed at the one or more electrodes. The method also includes outputting the set of three-dimensional positional data, the set of two-dimensional metric data, and the set of electrogram data from the memory to a display unit and displaying the set of three-dimensional positional data, the set of two-dimensional metric data, and the set of electrogram data on the display unit as a dynamic display and updating the dynamic display over time and displaying the updated dynamic display as a dynamic movie.

Alternatively or additionally to any of the embodiments above, the dynamic display includes a first panel graphically displaying the set of three-dimensional positional data.

Alternatively or additionally to any of the embodiments above, the first panel includes the set of three-dimensional positional data encoded with the set of metric data.

Alternatively or additionally to any of the embodiments above, the set of metric data is a set of activation times that are color-coded and warped onto the three-dimensional positional data.

Alternatively or additionally to any of the embodiments above, the dynamic display includes a second panel graphically displaying the set of metric data warped onto a two-dimensional grid.

Alternatively or additionally to any of the embodiments above, the second panel includes an interpolated activation map defined by the set of metric data.

Alternatively or additionally to any of the embodiments above, the second panel includes a set of conduction velocity vectors.

Alternatively or additionally to any of the embodiments above, the pre-determined metric includes activation times, fractional index, dominant frequency, amplitude, or combinations thereof.

Alternatively or additionally to any of the embodiments above, the dynamic display includes a third panel graphically displaying the set of electrogram data.

Alternatively or additionally to any of the embodiments above, wherein the third panel includes a time-amplitude plot of the set of electrogram data.

Alternatively or additionally to any of the embodiments above, wherein the time-amplitude plot is encoded with the set of metric data.

Alternatively or additionally to any of the embodiments above, the set of metric data includes activation times that are color-coded and mapped onto the time-amplitude plot.

Alternatively or additionally to any of the embodiments above, the dynamic display includes one or more additional panels graphically displaying an additional set of data.

An example method for displaying cardiac mapping data is disclosed. The method includes storing a set of three-dimensional positional data on a memory. The set of three-dimensional positional data corresponds to a location of one or more electrodes of a constellation catheter within a heart chamber. The method also includes storing a set of metric data on the memory. The set of metric data corresponds to one or more of activation times, fractional index, dominant frequency, or amplitude. The method also includes storing a set of electrogram data on the memory. The set of electrogram data corresponds to electrical activity sensed at the one or more electrodes. The method also includes outputting the set of three-dimensional positional data, the set of two-dimensional metric data, and the set of electrogram data from the memory to a display unit, simultaneously displaying the set of three-dimensional positional data, the set of two-dimensional metric data, and the set of electrogram data on separate regions of the display unit to define a dynamic display, and updating the dynamic display over time so as to dynamically convey at least the set of electrogram data.

Alternatively or additionally to any of the embodiments above, the dynamic display includes a first region graphically displaying the set of three-dimensional positional data encoded with the set of metric data.

Alternatively or additionally to any of the embodiments above, the dynamic display includes a second region graphically displaying the set of metric data as an interpolated activation map or a set of conduction velocity vectors.

Alternatively or additionally to any of the embodiments above, the dynamic display includes a third region graphically displaying the set of electrogram data as a time-amplitude plot.

Alternatively or additionally to any of the embodiments above, the dynamic display includes one or more additional regions graphically displaying an additional set of data.

An example system for cardiac mapping is disclosed. The system includes a catheter shaft with a plurality of electrodes coupled thereto. A processor is coupled to the catheter shaft. The processor is capable of storing a set of three-dimensional positional data on a memory, storing a set of metric data on the memory, storing a set of electrogram data on the memory, outputting the set of three-dimensional positional data, the set of two-dimensional metric data, and the set of electrogram data from the memory to a display unit, simultaneously displaying the set of three-dimensional positional data, the set of two-dimensional metric data, and the set of electrogram data on separate regions of the display unit to define a dynamic display, and updating the dynamic display over time so as to dynamically convey at least the set of electrogram data. The set of three-dimensional positional data corresponds to a location of the plurality of electrodes within a heart chamber. The set of metric data corresponds to one or more of activation times, fractional index, dominant frequency, or amplitude. The set of electrogram data corresponds to electrical activity sensed at the one or more electrodes.

Alternatively or additionally to any of the embodiments above, the dynamic display includes a first region graphically displaying the set of three-dimensional positional data encoded with the set of metric data, a second region graphically displaying the set of metric data as an interpolated activation map and/or a set of conduction velocity vectors, and a third region graphically displaying the set of electrogram data as a time-amplitude plot.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an example catheter system for accessing a targeted tissue region in the body for diagnostic and/or therapeutic purposes;

FIG. 2 is a side view of an example mapping catheter;

FIG. 3 is a schematic view of an example basket structure;

FIG. 4 is an illustration of an example activation map displaying known and unknown activation times;

FIG. 5 is a schematic representation of an example dynamic display;

FIG. 6 is illustrates an example dynamic display; and

FIG. 7 is a schematic representation of a dynamic movie.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Mapping the electrophysiology of heart rhythm disorders often involves the introduction of a constellation catheter or other mapping/sensing device having a plurality of electrodes and/or sensors (e.g., CONSTELLATION®, commercially available from Boston Scientific) into a cardiac chamber. The sensors detect the electric activity of the heart at sensor locations. It may be desirable to have the electric activity processed into electrogram signals that accurately represent cellular excitation through cardiac tissue relative to the sensor locations. A processing system may then analyze and output the signal to a display device.

Disclosed herein are methods for displaying physiological mapping data and dynamic displays. The dynamic display combines a static map (e.g., which may convey the instantaneous relationship of the information content with a color coded time-series display) with the three-dimensional locations of the electrodes on an integrated display. Furthermore, the dynamic display may be updated so as to dynamically display, for example, electrogram data as a dynamic movie. Some additional details regarding the methods are disclosed herein.

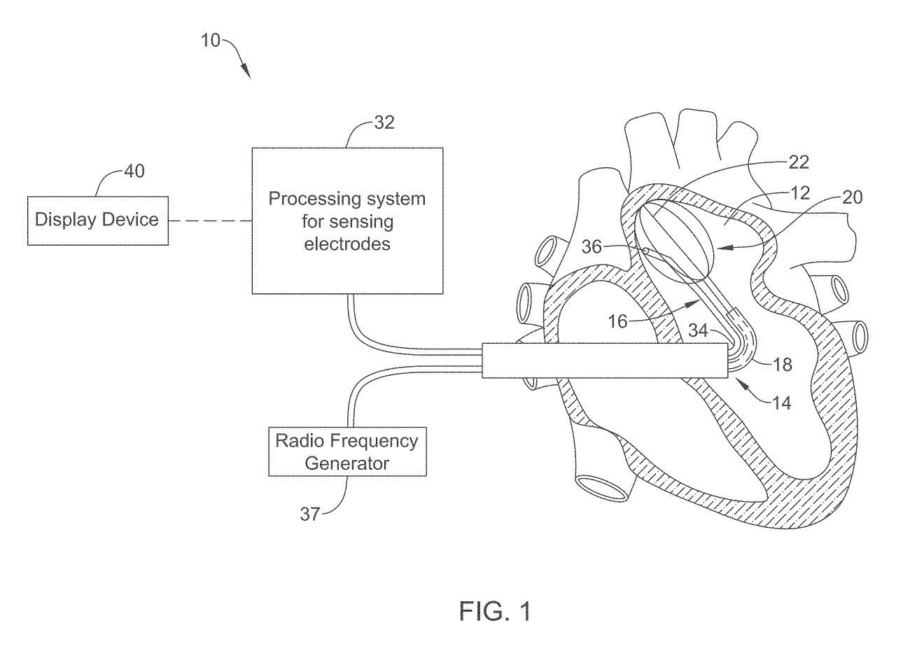

FIG. 1 is a schematic view of a system 10 for accessing a target region 12 in the body for diagnostic and/or therapeutic purposes. FIG. 1 generally shows system 10 deployed in the left atrium of the heart. Alternatively, system 10 can be deployed in other regions of the heart, such as the left ventricle, right atrium, or right ventricle. While the illustrated embodiment shows system 10 being used for mapping and/or ablating myocardial tissue, system 10 (and the methods described herein) may alternatively be configured for use in other tissue mapping and/or ablation applications, such as procedures for ablating or otherwise involving tissue in the prostrate, brain, gall bladder, uterus, nerves, blood vessels and other regions of the body, including in systems that are not necessarily catheter-based.

System 10 may include a mapping catheter 14 and an ablation catheter 16. Each probe 14/16 may be separately introduced into target region 12 through a vein or artery (e.g., the femoral vein or artery) using a suitable percutaneous access technique. Alternatively, mapping catheter 14 and ablation catheter 16 can be assembled in an integrated structure for simultaneous introduction and deployment in target region 12.

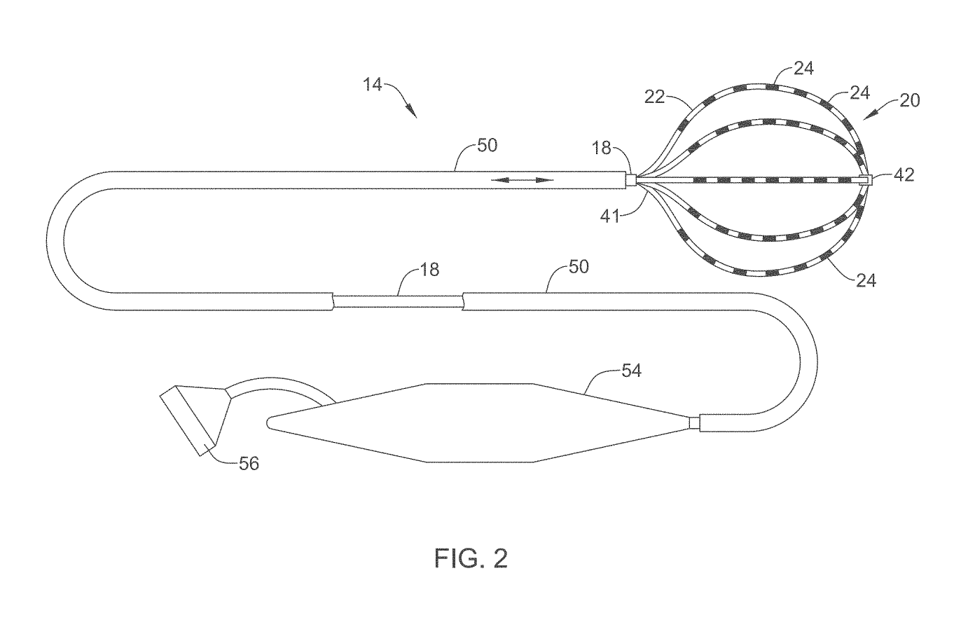

Mapping catheter 14 may include a catheter shaft 18. The distal end of the catheter shaft 18 may include a three-dimensional multiple electrode structure 20. Structure 20 may take the form of a basket having a plurality of struts 22 (see FIG. 2), although other multiple electrode structures could be used. A plurality of mapping electrodes 24 (not explicitly shown on FIG. 1, but shown on FIG. 2) may be disposed along struts 22. Each electrode 24 may be configured to sense intrinsic physiological activity in the anatomical region. In some embodiments, electrodes 24 may be configured to detect activation signals of the intrinsic physiological activity within the anatomical structure (e.g., the activation times of cardiac activity).

Electrodes 24 may be electrically coupled to a processing system 32. A signal wire (not shown) may be electrically coupled to each electrode 24 on basket structure 20. The wires may extend through shaft 18 and electrically couple each electrode 24 to an input of processing system 32. Electrodes 24 may sense electrical activity in the anatomical region (e.g., myocardial tissue). The sensed activity (e.g., activation signals) may be processed by processing system 32, which may assist the physician by generating an electrical activity map (e.g., a vector field map, an activation time map, etc.) to identify the site or sites within the heart appropriate for a diagnostic and/or treatment procedure. For example, processing system 32 may identify a near-field signal component (e.g., activation signals originating from cellular tissue adjacent to the mapping electrode 24) or from an obstructive far-field signal component (e.g., activation signals originating from non-adjacent tissue). The near-field signal component may include activation signals originating from atrial myocardial tissue whereas the far-field signal component may include activation signals originating from ventricular myocardial tissue. The near-field activation signal component may be further analyzed to find the presence of a pathology and to determine a location suitable for ablation for treatment of the pathology (e.g., ablation therapy).

Processing system 32 may include dedicated circuitry (e.g., discrete logic elements and one or more microcontrollers; a memory or one or more memory units, application-specific integrated circuits (ASICs); and/or specially configured programmable devices, such as, for example, programmable logic devices (PLDs) or field programmable gate arrays (FPGAs)) for receiving and/or processing the acquired activation signals. In at least some embodiments, processing system 32 includes a general purpose microprocessor and/or a specialized microprocessor (e.g., a digital signal processor, or DSP, which may be optimized for processing activation signals) that executes instructions to receive, analyze and display information associated with the received activation signals. In such implementations, processing system 32 can include program instructions, which when executed, perform part of the signal processing. Program instructions can include, for example, firmware, microcode or application code that is executed by microprocessors or microcontrollers. The above-mentioned implementations are merely exemplary. A variety of processing systems 32 are contemplated.

In some embodiments, processing system 32 may be configured to measure the electrical activity in the myocardial tissue adjacent to electrodes 24. For example, in some embodiments, processing system 32 may be configured to detect electrical activity associated with a dominant rotor or divergent activation pattern in the anatomical feature being mapped. Dominant rotors and/or divergent activation patterns may have a role in the initiation and maintenance of atrial fibrillation, and ablation of the rotor path, rotor core, and/or divergent foci may be effective in terminating the atrial fibrillation. In either situation, processing system 32 processes the sensed activation signals to generate a display of relevant characteristics, such as an isochronal map, activation time map, action potential duration (APD) map, a vector field map, a contour map, a reliability map, an electrogram, a cardiac action potential, and/or the like. The relevant characteristics may be used by the physician to identify a site suitable for ablation therapy.

Ablation catheter 16 may include a flexible catheter body 34 that carries one or more ablation electrodes 36. Electrodes 36 may be electrically connected to a radio frequency (RF) generator 37 (or other suitable energy source) that is configured to deliver ablation energy to electrodes 36. Ablation catheter 16 may be movable with respect to the anatomical feature to be treated, as well as the structure 20. Ablation catheter 16 may be positionable between or adjacent to electrodes 24 of structure 20, for example, when the one or more ablation electrodes 36 are positioned adjacent to target region 12.

Processing system 32 may output data to a suitable output or display device 40, which may display relevant information for a clinician. Device 40 may be a CRT, LED, or other type of display, a printer, or the like. Device 40 may be utilized to present the relevant characteristics in a format most useful to the physician. In addition, processing system 32 may generate position-identifying output for display on device 40 that aids the physician in guiding ablation electrode(s) 36 into contact with tissue at the site identified for ablation.

Turning now to FIG. 2, here some of the features of mapping catheter 14 can be seen. For example, FIG. 2 illustrates that structure 20 an end cap 42 between which struts 22 generally extend in a circumferentially spaced relationship. Struts 22 may be made of a resilient inert material, such as Nitinol, other metals, silicone rubber, suitable polymers, or the like and extend between a base region 41 and end cap 42 in a resilient, pretensioned condition, to bend and conform to the tissue surface they contact. In some embodiments, eight struts 22 may form structure 20. Additional or fewer struts 22 could be used in other embodiments. As illustrated, each strut 22 may carry eight mapping electrodes 24. Additional or fewer mapping electrodes 24 could be disposed on each strut 22 in other embodiments. Various dimensions are contemplated for structure. For example, structure 20 may be relatively small (e.g., 40 mm or less in diameter). In alternative embodiments, structure 20 may be smaller or larger (e.g., 40 mm in diameter or greater).

A slidable sheath 50 may be movable along the major axis of shaft 18. Moving sheath 50 distally relative to shaft 18 may cause sheath 50 to move over structure 20, thereby collapsing the structure 20 into a compact, low profile condition suitable for introduction into and/or removal from an interior space of an anatomical structure, such as, for example, the heart. In contrast, moving the sheath 50 proximally relative to shaft 18 may expose structure 20, allowing structure 20 to elastically expand and assume the basket configuration illustrated in FIG. 2.

A signal wire (not shown) may be electrically coupled to each mapping electrode 24. The wires may extend through shaft 18 of mapping catheter 20 (or otherwise through and/or along shaft 18) into a handle 54, in which they are coupled to an external connector 56, which may be a multiple pin connector. Connector 56 may electrically couple mapping electrodes 24 to processing system 32. These are just examples. Some addition details regarding these and other example mapping systems and methods for processing signals generated by the mapping catheter can be found in U.S. Pat. Nos. 6,070,094, 6,233,491, and 6,735,465, the disclosures of which are hereby expressly incorporated herein by reference.

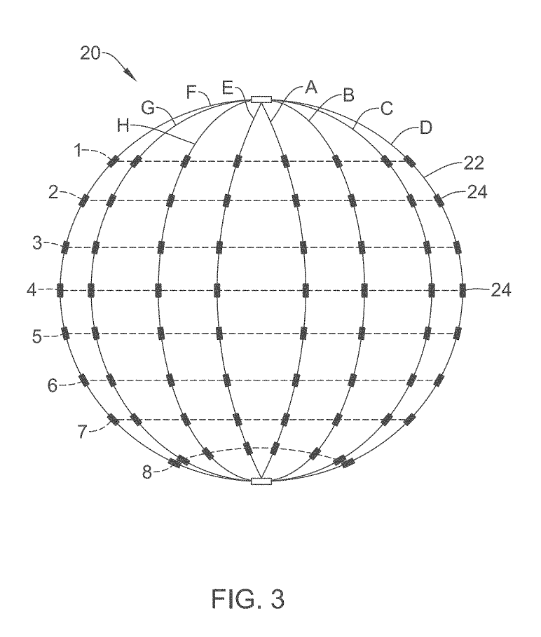

To illustrate the operation of the system 10, FIG. 3 is a schematic side view of basket structure 20. In the illustrated embodiment, basket structure includes 64 mapping electrodes 24. Electrodes 24 may be disposed in groups of eight electrodes (labeled 1, 2, 3, 4, 5, 6, 7, and 8) on each of eight struts 22 (labeled A, B, C, D, E, F, G, and H). While an arrangement of sixty-four mapping electrodes 24 is shown disposed on basket structure 20, mapping electrodes 24 may alternatively be arranged in different numbers (more or fewer splines and/or electrodes), on different structures, and/or in different positions. In addition, multiple basket structures can be deployed in the same or different anatomical structures to simultaneously obtain signals from different anatomical structures.

When basket structure 20 is positioned adjacent to the anatomical structure to be treated (e.g. left atrium, left ventricle, right atrium, or right ventricle of the heart), processing system 32 may be configured to record the activation signals from each electrode 24 channel related to physiological activity of the anatomical structure (e.g., the electrodes 24 measure electrical activation signals associated with the physiology of the anatomical structure). The activation signals of physiological activity may be sensed in response to intrinsic physiological activity or based on a predetermined pacing protocol instituted by at least one of the plurality of electrodes 24.

Electrodes 24 that contact healthy, responsive cellular tissue may sense a change in the voltage potential of a propagating cellular activation wavefront. Further, in a normal functioning heart, electrical discharge of the myocardial cells may occur in a systematic, linear fashion. Therefore, detection of non-linear propagation of the cellular excitation wavefront may be indicative of cellular firing in an abnormal fashion. For example, cellular firing in a rotating pattern may indicate the presence of dominant rotors and/or divergent activation patterns. Further, because the presence of the abnormal cellular firing may occur over localized target tissue regions, it is possible that electrical activity may change form, strength or direction when propagating around, within, among or adjacent to diseased or abnormal cellular tissue. Identification of these localized areas of diseased or abnormal tissue may provide a clinician with a location for which to perform a therapeutic and/or diagnostic procedure. For example, identification of an area including reentrant or rotor currents may be indicative of an area of diseased or abnormal cellular tissue. The diseased or abnormal cellular tissue may be targeted for an ablative procedure.

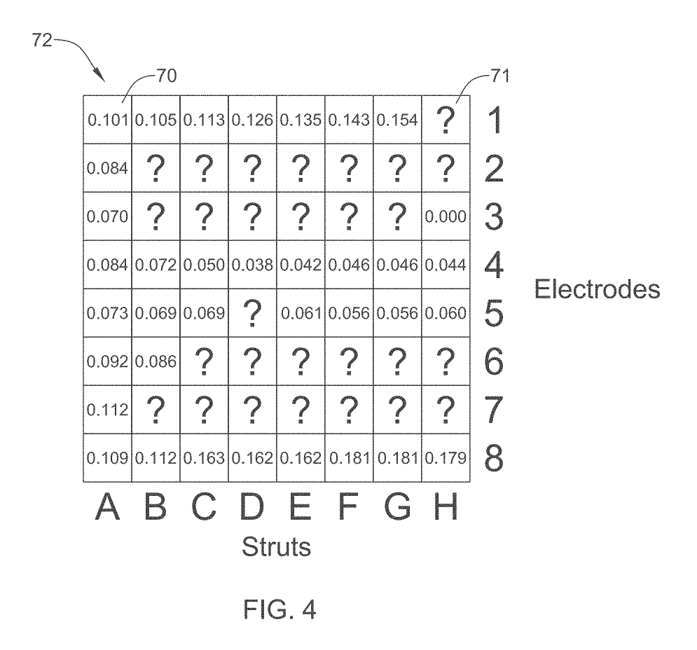

FIG. 4 illustrates an example activation map 72 showing activation times sensed by electrodes 24. Activation map 72 may include a two-dimensional grid that visually represents mapping electrodes 24. For example, activation map 72 may include an 8×8 matrix displaying sixty-four (64) electrode spaces that represent the sixty-four (64) electrodes on a constellation catheter or similar sensing device. Mapping electrodes 24 may be organized and/or identified by electrode number (e.g. electrodes 1-8) and spline location (e.g. splines A-H). Other combinations of electrodes and/or splines are contemplated.

The activation time for an electrode 24 may be defined as the time elapsed between an activation “event” being sensed on a target mapping electrode 24 and a reference electrode. For example, a space 70 on map 72 representing electrode 1 on strut A displays an activation time of 0.101 ms. However, it is possible that one or more electrodes 24 will be unable to sense and/or collect an activation time. For example, one or more spaces like a space 71 representing electrode 1 on spline H may display a “?.” The “?” may indicate that the particular electrode corresponding to that location on the multiple electrode structure 20 cannot sense an activation time. Therefore, the “?” may represent missing signal data. Missing signal data and/or an incomplete activation map may prevent the identification of diseased or abnormal cellular tissue.

Some embodiments may include generating a color map corresponding to activation map 72. Each unique activation time may be assigned a unique, differentiating color. It is contemplated that a variety of color combinations may be included in generating the color-based activation time map. Further, the color map may be displayed on a display. Additionally, the color map may help a clinician identify the propagation direction of cellular firing. Activation map 72 may display an activation time or color for known signals and not display an activation time or color for unknown and/or missing activation time data. The use of color to differentiate activation times is just an example. It is contemplated that other means may be used to differentiate activation times. For example, texture, symbols, numbers, or the like may be used as differentiating characteristics.

In order to maximize the utility of activation map 72, it may be desirable to populate unknown activation times. Therefore, in some embodiments it may be desirable to interpolate activation times for missing signal data and populate and/or fill in the activation time map 72 accordingly. In practice, it may be that electrodes 24 in close proximity to one another will experience similar cellular events (e.g. depolarization). For example, as a cellular activation wavefront propagates across an atrial surface, electrodes 24 in close proximity to one another will likely experience similar cellular activation times. Therefore, when selecting an interpolation method, it may be desirable to select a method that incorporates the relative distance between neighboring electrodes and utilizes those distances in an algorithm to estimate unknown data points. One method to interpolate activation times and thereby fill in missing electrode data is to utilize an interpolation method that estimates the missing electrode data based on the electrode's relationship and/or proximity to known electrode data. The method may include identifying the physical position of all electrodes 24 in three-dimensional space, determining the distance between electrodes 24, and interpolating and/or estimating the missing electrode values. The estimated values may then be used to populate diagnostic displays (e.g. activation map). Therefore, the interpolation method may include any interpolation method that incorporates neighboring electrode information (e.g. distance between electrodes) in its estimation algorithm. Example interpolation methods may include Radial Basis Function (RBF) and/or Kriging interpolation. These are only examples. It is contemplated that other interpolation methods that incorporate neighboring data point information may be utilized with the embodiments disclosed herein.

As suggested herein, data collected or sensed by electrodes 24 can be collected, stored, or otherwise “processed” by processing system 32. This may include storing data on one or more memories within processing system 32 and/or system 10. The data may help a clinician assess, diagnose, and/or treat a patient. In order for the data to be efficiently utilized, the data may be processed and/or displayed on display device 40. However, in some instances, time-series information gathered from multiple points on a three-dimensional surface may be hard to visualize. Therefore, it may be desirable to combine spatio-temporal information into an integrated display (e.g., a dynamic display) that provides a variety of information such as, for example, three-dimensional locations of electrodes 24, activation maps/times, conduction velocity vectors, electrogram information, or the like.

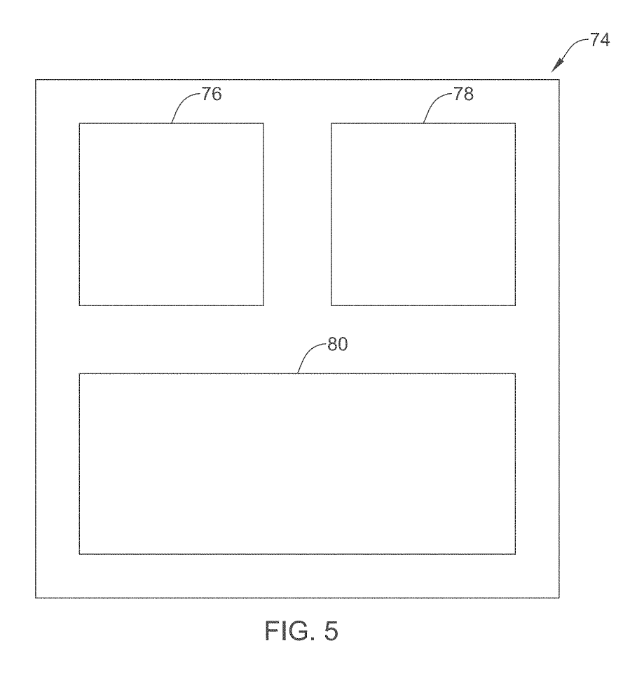

FIG. 5 schematically illustrates an example dynamic display 74. Display 74 may include a plurality of panels such as a first panel 76, a second panel 78, and a third panel 80. Display 74 may be output or otherwise “displayed” on display device 40 where it can be visualized by a clinician. For example, each panel 76/78/80 may provide a clinician with useful information that may aid in assessing, diagnosing, and/or treating a patient. It can be appreciated that the number of panels, the size and/or shape of the panels, and the like may vary.

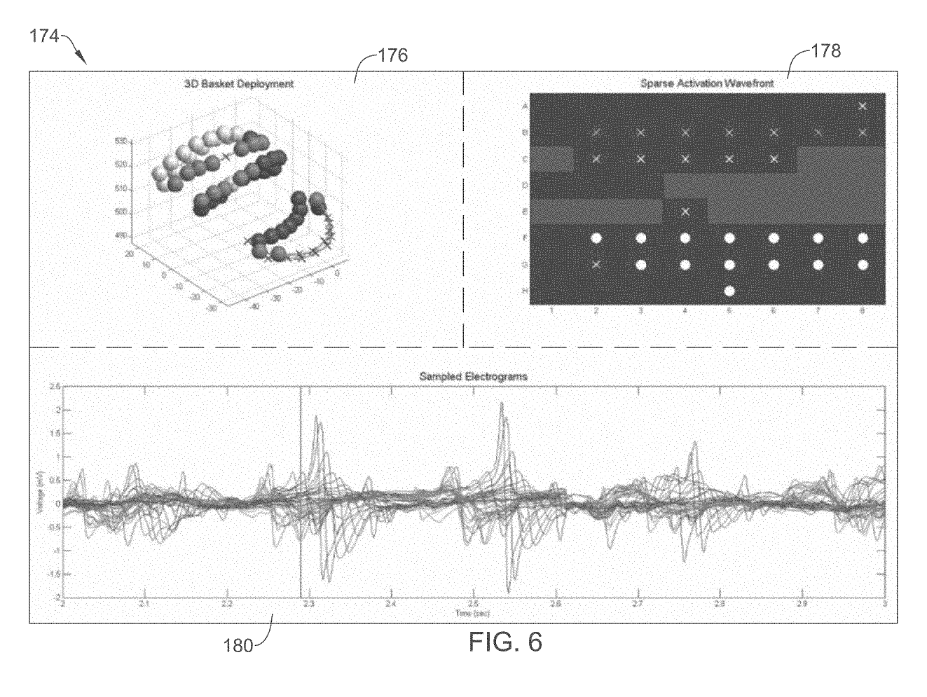

FIG. 6 illustrates an example display 174 with example graphical representations of data shown in panels 176/178/180. Each graphical representation may be formed by collecting or sensing a physiological parameter by electrodes 24 (and/or electrodes 36), transmitting the collection or “set” of raw data to processing system 32, storing the set of data on a memory (e.g., a memory that may be part of processing system 32), processing the data so that it can be utilized or output in a desirable manner, and outputting the processed data to display device 40. Multiple sets of data can be output and displayed on dynamic display 174 in each of panels 176/178/180. In this example, panel 176 may include a graphical representation of three-dimensional positional data corresponding to a location of electrodes 24 within a body chamber (e.g., target region 12). The graphical representation takes the form of a three-dimensional graph where example electrodes 24 are shown as spheres distributed on the graph.

While this graphical representation shown in panel 176 may include positional data, other data may also be included and graphically represented. For example, electrodes 24 may collect or sense additional data such as a set of metric data. The metric data may be data such as activation times, fractional index, dominant frequency, amplitude, or the like. In this example, metric data corresponding to activation times may be added to or otherwise warped onto the graphical display in panel 176. For example, activation times sensed at electrodes 24 may be graphically represented on the three-dimensional graph by color coding the various spheres on the graph. In other words, each sphere may be color coded so that not only is the position of electrodes 24 shown, the color may correspond to an activation time sensed at each electrode 24. While color coding may be convenient manner to warp metric data onto the three-dimensional graph, other methods may also be utilized such as texturing, patterning, or the like.

It should be noted that in some instances, other graphics may be added to or otherwise shown in any of the panels such as panels 176/178. For example, some of electrodes 24 are represented on the three-dimensional graph in panel 176 with an “X”, which may indicate that there was no contact between that particular electrode and the target tissue. Similarly, in panel 178, some boxes may include a central “X”, which may indicate that the metric data represented in the box (e.g., activation time) is an interpolated value. Furthermore, some boxes in panel 178 may include a central “solid white dot”, which may represent that electrical signal was present but the metric data was not extracted such that the metric data represented in the box is an interpolated value.

Panel 178 may provide a graphical representation of additional data. For example, a set of metric data or other data collected by electrodes 24 and output to panel 178. In this example, the metric data may be activation times and the activation times may be displayed on a two-dimensional grid or sparse activation wavefront map. The sparse activation wavefront map may be color-coded, textured, patterned, or the like so as to convey the desired information to a clinician.

Panel 180 may provide a graphical representation of electrical activity sensed at electrodes 24 in the form of an electrogram or electrogram data. A time-amplitude (e.g., a time-voltage) plot of the set of electrogram data may be shown in panel 180. This may allow a clinician to visualize the electrical activity sensed at electrodes 24 over time.

While this graphical representation shown in panel 180 may include electrogram data, other data may also be included and graphically represented. For example, electrodes 24 may collect or sense additional data such as a set of metric data. The metric data may be data such as activation times, fractional index, dominant frequency, amplitude, or the like. In this example, metric data corresponding to activation times may be added to or otherwise warped onto the graphical display in panel 180. For example, activation times sensed at electrodes 24 may be graphically represented on the time-amplitude plot by color coding the individual time-amplitude traces on the plot. In other words, each electrogram may be color coded so that not only is the time-voltage relation of electrodes 24 shown, the color may correspond to an activation time sensed at each electrode 24. While color coding may be convenient manner to map metric data onto the time-amplitude plot, other methods may also be utilized.

Collectively, display 174 may be shown on display device 40 to convey desirable information to the clinician. At least some of panels 176/178/180 may be dynamically updated over time. For example, at least panel 180 may be dynamically updated. In some embodiments, each panel 176/178/180 may updated over time so that real-time information can be displayed. By displaying multiple pieces of information in this format, a clinician may be able to more easily assess, diagnose, and/or treat a patient in an efficient manner.

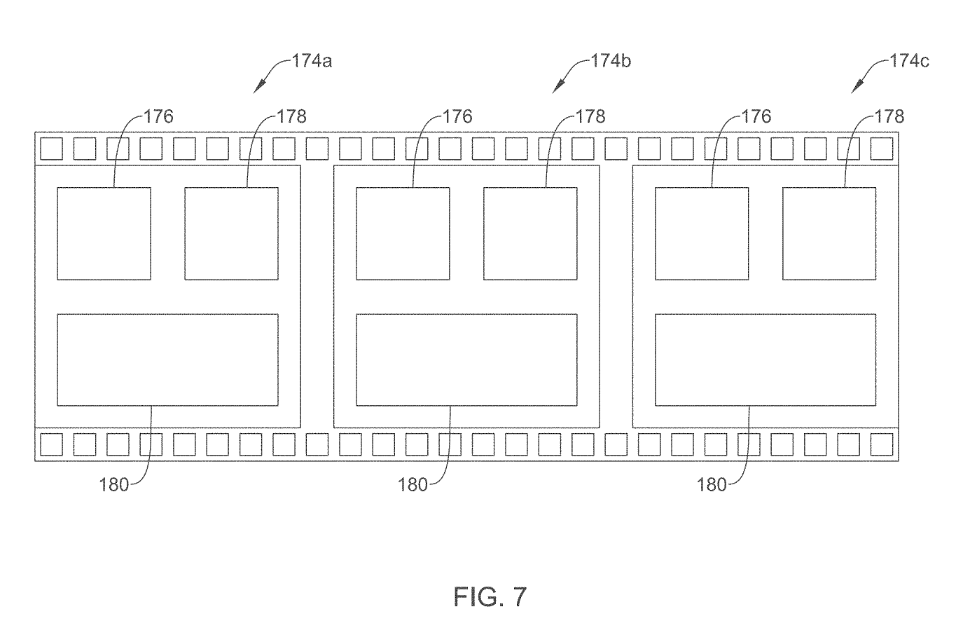

FIG. 7 schematically illustrates how display 174 may be updated over time and displayed as a dynamic movie. In this illustration, a first display or frame 174a of the movie may graphically display useful data in panels 176/178/180. Subsequent displays or frames 174a/174c/etc. may further graphically display useful data at differing points in time. Display 174 may continually update so that the movie may provide a clinician with real-time graphical representations of data so as to more efficient assess, diagnose, and/or treat a patient.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A computer-implemented method for displaying physiological mapping data, the method comprising:
receiving cardiac metric data sensed by a plurality of electrodes of a catheter during a period of time, the cardiac metric data including a respective electrogram sensed by each of the plurality of electrodes during the period of time and positional data of the plurality of electrodes when the cardiac metric data is being sensed by the plurality of electrodes;
outputting to a first panel of a display the respective electrogram sensed by each of the plurality of electrodes at a first time of the period of time;
outputting to a second panel of the display a representation of the catheter and the plurality of electrodes at the first time, the representation of the catheter and the plurality of electrodes at the first time including the positional data of the plurality of electrodes at the first time;
updating the first panel of the display to display the respective electrogram sensed by each of the plurality of electrodes at a second time of the period of time, the first time being different than the second time; and
updating the second panel of the display to display the representation of the catheter and the plurality of electrodes at the second time, the representation of the catheter and the plurality of electrodes at the second time including the positional data of the plurality of electrodes at the second time.

2. The computer-implemented method of claim 1, wherein to output to a first panel of a display the respective electrogram sensed by each of the plurality of electrodes at a first time, the method comprises identifying each respective electrogram with an identifier corresponding to an electrode of the plurality of electrodes that sensed the respective electrogram.

3. The computer-implemented method of claim 2, wherein the identifier includes a spine identifier of the catheter on which the electrode is located and a position identifier corresponding to the electrode position on the spine.

4. The computer-implemented method of claim 1, wherein to output to the second panel of the display the representation of the catheter and the plurality of electrodes at the first time including the positional data of the plurality of electrodes at the first time, the method comprises including color coding on the representation of each of the plurality of electrodes corresponding to an activation time sensed by each of the plurality of electrodes.

5. The computer-implemented method of claim 4, wherein the activation time sensed by each of the plurality of electrodes is determined in reference to a reference electrode of the plurality of electrodes.

6. The computer-implemented method of claim 4, wherein one or more electrodes of an additional plurality of electrodes of the catheter did not sense cardiac metric data at the first time and wherein the one or more electrodes of the additional plurality of electrodes are color coded with a differentiating color.

7. The computer-implemented method of claim 1, wherein one or more electrodes of an additional plurality of electrodes of the catheter did not sense cardiac metric data at the first time, and wherein the method further comprises interpolating cardiac metric data for the one or more electrodes of the additional plurality of electrodes at the first time.

8. The computer-implemented method of claim 1, further comprising outputting to a third panel of the display cardiac metric data that is not presented in either the first panel or the second panel.

9. The computer-implemented method of claim 8, wherein the third panel includes an interpolated activation map.

10. The computer-implemented method of claim 8, wherein the third panel includes a set of conduction velocity vectors.

11. The computer-implemented method of claim 1, wherein the cardiac metric data includes at least one of: an activation time, a fractional index, a dominant frequency, and an amplitude.

12. A system for cardiac mapping, the system comprising:
a catheter shaft comprising a plurality of electrodes coupled to a distal end of the catheter shaft, the plurality of electrodes configured to sense cardiac metric data during a period of time, the cardiac metric data including a respective electrogram sensed by each of the plurality of electrodes during the period of time and positional data of the plurality of electrodes when the cardiac metric data is being sensed by the plurality of electrodes;
a processor communicatively coupled to the plurality of electrodes, the processor being configured to:
receive the cardiac metric data;
output to a first panel of a display a representation of the catheter and the plurality of electrodes at a first time of the period of time, the representation of the catheter and the plurality of electrodes at the first time including the positional data of the plurality of electrodes at the first time and including color coding on each of the plurality of electrodes corresponding to an activation time sensed by each of the plurality of electrodes at the first time; and
update the first panel of the display to display a representation of the catheter and the plurality of electrodes at a second time of the period of time, the representation of the catheter and the plurality of electrodes at the second time including the positional data of the plurality of electrodes at the second time and including color coding on each of the plurality of electrodes corresponding to an activation time sensed by each of the plurality of electrodes at the second time, the first time being different than the second time.

13. The system of claim 12, wherein the activation time sensed by each of the plurality of electrodes is determined in reference to a reference electrode of the plurality of electrodes.

14. The system of claim 12, wherein one or more electrodes of an additional plurality of electrodes of the catheter did not sense cardiac metric data at the first time and wherein the representation of the catheter and the plurality of electrodes includes a differentiating color code for the one or more electrodes of the additional plurality of electrodes.

15. The system of claim 12, wherein one or more electrodes of an additional plurality of electrodes of the catheter did not sense cardiac metric data at the first time, and wherein the processor is further configured to interpolate cardiac metric data of the one or more electrodes of the additional plurality of electrodes at the first time.

16. The system of claim 12, the processor further configured to:
output to a second panel of a display the respective electrogram data sensed by each of the plurality of electrodes at the first time; and
update the first panel of the display to display the respective electrogram data sensed by each of the plurality of electrodes at the second time.

17. The system of claim 16, wherein to output to a first panel of a display the respective electrogram data sensed by each of the plurality of electrodes at a first time, the processor is configured to identify each respective electrogram data with an identifier corresponding to an electrode of the plurality of electrodes that sensed the respective electrogram data.

18. The system of claim 17, wherein the identifier includes a spine identifier of the catheter on which the electrode is located and a position identifier corresponding to the electrode position on the spine.

19. The system of claim 12, the processor being further configured to output to a third panel of the display cardiac metric data that is not presented in either the first panel or the second panel.

20. The system of claim 19, wherein the third panel includes an interpolated activation map.
